(19) United States
(12) Patent Application Publication
(10) Pub. No.: US 2011/0087166 A1
(43) Pub. Date: Apr. 14, 2011

(54) LABORATORY SYRINGE PUMPS

(76) Inventors: Mark Davis, Millville, MA (US); Richard K. Bennett, Winchester, NH (US); Bruce H. Edwards, Marlborough, MA (US); Andrew J. McKenna, Warwick, RI (US); Justin M. Piccirillo, Uxbridge, MA (US); Ron Y. Sostek, Newton, MA (US)

(21) Appl. No.: 12/755,245

(22) Filed: Apr. 6, 2010

Related U.S. Application Data
(60) Provisional application No. 61/167,071, filed on Apr. 6, 2009.

(51) Int. Cl. A61M 37/00

(52) U.S. Cl. 604/155

(57) ABSTRACT

A syringe pump which includes a pusher block is slidably mounted on bearings on one or more guide rails. The pusher block is advanced or retracted by a lead screw having an axis which is offset from the axis of the one or more guide rails which are supported at their respective ends by support blocks, wherein forward and/or trailing sides of the pusher block bearings are extended, and wherein the guide rail support blocks include recesses for accommodating the extended ends of the pusher block slide bearings. In another embodiment, the syringe pump includes quick connect-disconnect fixtures for loading and unloading syringes. Other mechanical and programmable improvements are described in the specification.

### CROSS REFERENCE TO RELATED APPLICATION

This application claims priority from U.S. Provisional Application Ser. No. 61/167,071 filed Apr. 6, 2009.

### FIELD OF THE INVENTION

The present invention relates generally to syringe pumps, and more particularly improvements in syringe pumps. The invention has particular utility in connection with syringe pumps for laboratory or general fluidics transfer of gas and liquids use, e.g. for conducting small scale chemical reactions, liquid sample preparation for high pressure liquid chromatography, as well as biotechnological and pharmaceutical liquid handing and assay procedures, animal infusion and microfluidics and to deliver flow to pulse sensitive de-factors and will be described in connection with such procedures, although other utilities are contemplated.

### BACKGROUND OF THE PRESENT INVENTION

In small-scale chemical reactions, (e.g. for chemical and biochemical laboratory research), the controlled addition of reagents (liquids or gases) to a chemical reaction often is accomplished by the use of disposable syringes. A researcher may choose to fill a disposable plastic, glass or stainless steel syringe with a desired reagent and then control the rate of addition of the reagent into a reaction mixture, or into a chromatography column, for example. Syringes are also employed for the controlled introduction of a pharmaceutical or nutrient to a laboratory animal or other biological system. Ideally introduction of reagents or pharmaceuticals should be controlled accurately and reproducibly over time.

While introduction of reagents or pharmaceuticals from a syringe can be accomplished manually, the art has developed automated, programmable syringe pumps for introducing reagents or pharmaceuticals into a chemical reaction or biological system. With the introduction of automated, programmable syringe pumps, operators were freed of “hands-on” syringe operation, freeing the laboratory worker to do other things, while at the same time increasing accuracy and reproducibility of experiments.

A state of the art syringe pump 20 is illustrated in FIG. 1. Referring to FIG. 1, syringe pump 20 employs a stepping motor or AC motor(not shown) to turn a lead screw 22 and its mating nut or half nut (not shown) to drive the plunger of a syringe 30. The lead screw/stepping motor combination allows for a simple, digitally-controlled open-loop plunger drive mechanism pusher block 26, and has a resolution defined by the number of steps and/or microsteps per revolution of the stepping motor, pulley or gear ratio, and the pitch of the lead screw. The pusher block carriage assembly 26 rides on one or a plurality of guide rails 28. In a conventional syringe pump, such as illustrated in FIG. 1, the axis of the lead screw 22 which connects to the pusher block 26, is offset from the axis of the syringe plunger 30. Such offset may cause slight and undesirable non-linear motion or mechanical linkages that connect the syringe plunger 30 to the lead screw 22, which results in pulsatile deliveries. Conventional syringe pumps also have substantial hysteresis, which limits their accuracy and reproducibility.

Additionally, current state of the art syringe pumps require significant set-up time, e.g., to accommodate different size syringes and/or to change syringes, and also leave much to be desired in terms of ease of use.

### SUMMARY OF THE INVENTION

An object of the present invention is to overcome the aforesaid and other disadvantages of currently available, manual or automated programmable syringe pumps. In order to achieve this objective, a detailed study was undertaken of both mechanical and programmable control features of current syringe pumps. And, as a result, several improvements were made in both mechanical and programmable control features as will be discussed below.

### BRIEF DESCRIPTION OF THE DRAWINGS

Further features and advantages of the present invention will be seen from the following detailed description, taken in conjunction with the accompanying drawings, wherein like numerals depict like parts, and wherein:

FIG. 1 is a perspective view of a prior art syringe pump;

FIG. 2 is a perspective view of an improved syringe pump made in accordance with the present invention;

FIGS. 3-4 are perspective views from various angles showing details of quick-connect/disconnect pusher block assemblies in accordance with the present invention;

FIGS. 5 and 6 are perspective views showing details of quick-connect/disconnect syringe barrel flange capture assemblies in accordance with the present invention;

FIG. 7 is a perspective view showing details of an adjustable holder and clamp assembly in accordance with the present invention;

FIG. 8 is a block diagram showing improvements in a programmable syringe pump system in accordance with the present invention;

FIG. 9 is a block flow diagram and 10 is a graphical representation of improved flow rate control in accordance with the present invention; and

FIG. 11 is a block diagram of a pumping control system in accordance with the present invention.

### MECHANICAL IMPROVEMENTS

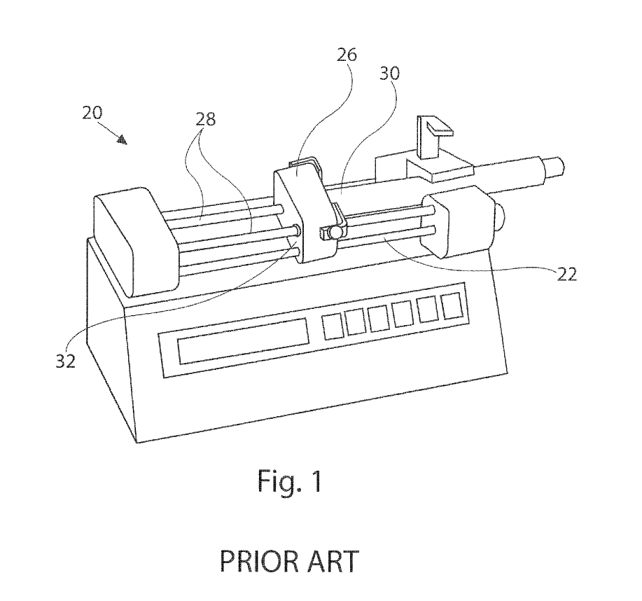

As noted supra, current syringe pumps suffer from undesirable non-linear motions of the mechanical linkage resulting in pulsatile deliveries. Referring again to FIG. 1, pump pulsative delivery is due, in part, to a torque on the pusher block assembly 26 as the lead screw 22 is advanced. The torque on the pusher block assembly 26 twists the pusher block assembly 26 on the guide rail 28. While lengthening the bearing support 32 of the assembly 26 would reduce the aforesaid torque problem, simply lengthening the bearing support 32 would reduce the length of the travel path resulting in incomplete emptying of the syringes carried thereon. And, while the guide rails 28 could be extended to accommodate longer bearing supports 32, that would require increasing the size and thus the footprint of the apparatus. Since the apparatus is designed for laboratory use, increasing the footprint of the apparatus is disadvantageous since laboratory bench and hood space is a premium.

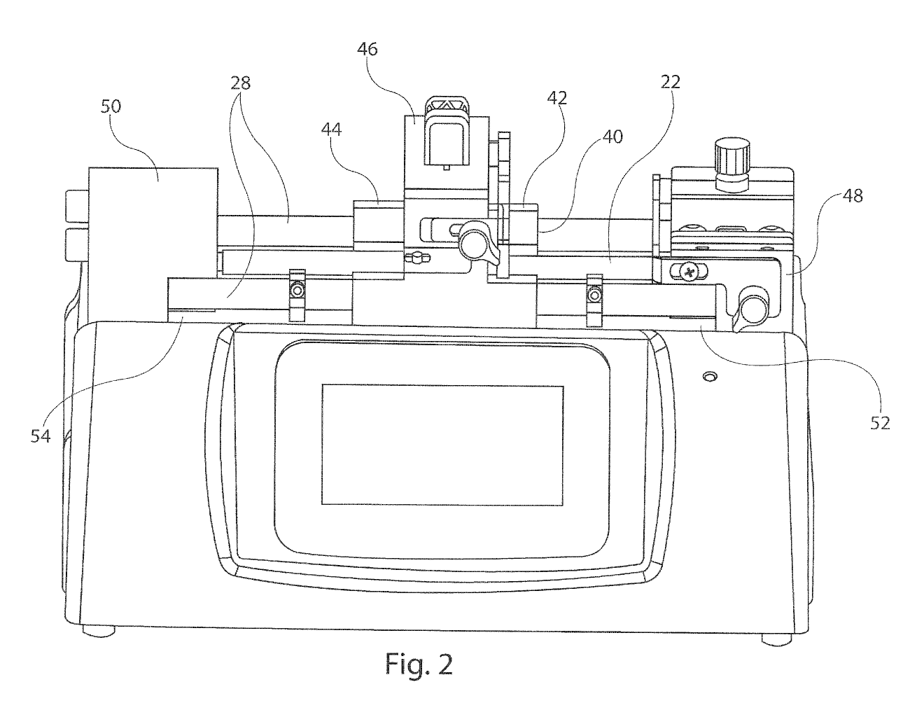

Referring to FIG. 2, in order to improve mechanical performance without increasing the footprint of the apparatus or contributing to pulsatility, the ends of the linear slide bearings 40 are lengthened at 42, 44 to extend beyond the forward and/or trailing sides of the pusher block 46. Extending the ends 42, 44 of the slide bearings 40 increases the length of the bearings which in turn increases resistance to non-linear motion of the mechanical linkage. In order to accommodate the increased length of the slide bearings, without increasing the overall size of the apparatus, the guide rail 28 support blocks 48, 50 are provided with recesses 52, 54 for accommodating the extended ends 42, 44 of the slide bearings. Extending the length of pusher block 46 increases the length of the bearings which in turn increases resistance to undesirable non-linear motions. With this arrangement, the linear guide or guide rails 28 are still supported and constrained by the end support blocks 48, 50. However, the pusher block 46 still is permitted to travel the entire working length of the lead screw 22, since at the fullest extent of travel, the extended bearing ends 42, 44 slide into the recesses 52, 54 in the support blocks 48, 50. The resulting structure is a substantially lengthened slide bearing which provides a significantly improved mechanical linkage far less susceptible to pulsatility, but without increasing the size of the syringe pump or its footprint.

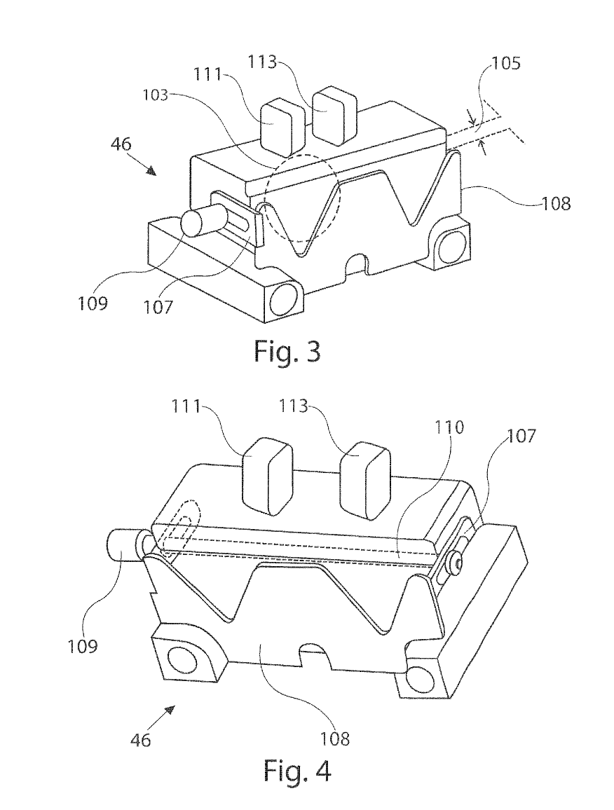

Referring also to FIGS. 3-6, another aspect of the invention provides a quick connect/disconnect which facilitates loading and unloading of syringes from a pump, eliminates the need for special tools to load and unload syringes from the pump, and eliminates the need for special tools to adjust for different size syringes. More particularly, as shown in FIGS. 3 and 4, the pusher block 46 includes a quick connect/disconnect flange capture bracket 108 for capturing the plunger flange when a syringe plunger 103 (shown in phantom in FIG. 3) is loaded on the pump. The plunger flange capture bracket 108 which is provided with tabs 107 which in turn are slidably mounted on a threaded shaft 110 that runs through pusher block 46. The distal end of the shaft 110 opposite the thumb-screw or knob 109 includes a flange and retaining clip or other similar device so that as the thumb-screw or knob 109 is turned, the side tabs 107 of the flange capture bracket 108 are pulled tightly against the pusher block 46, locking the assembly in place. The shaft 110 may be provided with an anti-rotation device to eliminate rotation of the shaft 110 as the thumb-screw or knob 109 is turned, thus further facilitating the quick connect/disconnect tool-free locking procedure. A thumb-screw or knob 109 permits a user to lock and unlock the shaft providing an adjustable gap 105 between the plunger flange capture bracket 108 and the syringe pusher block 46. Alternatively, knob 109 could be replaced by a rocker cam. The adjustable gap 105 may be set to fit a variety of syringe plunger flanges associated with different types and sizes of syringes available for use with the syringe pump. To capture a syringe plunger flange, the syringe plunger flange capture bracket 108 is first opened to permit a syringe plunger flange to be loaded on the pump. The bracket 108 is then pushed firmly against the syringe plunger flange, to close any gap between the syringe pusher block 46, the syringe plunger flange and the syringe plunger flange capture bracket 108. The syringe plunger flange capture bracket 108 is then locked by twisting thumb-screw or knob 109.

Syringe pusher block 46 is slidably adjusted along the lead screw 22 by squeezing release handles 111, 113 which unlock when squeezed. Release handles 111, 113 are conventional in syringe pumps and will not be further described.

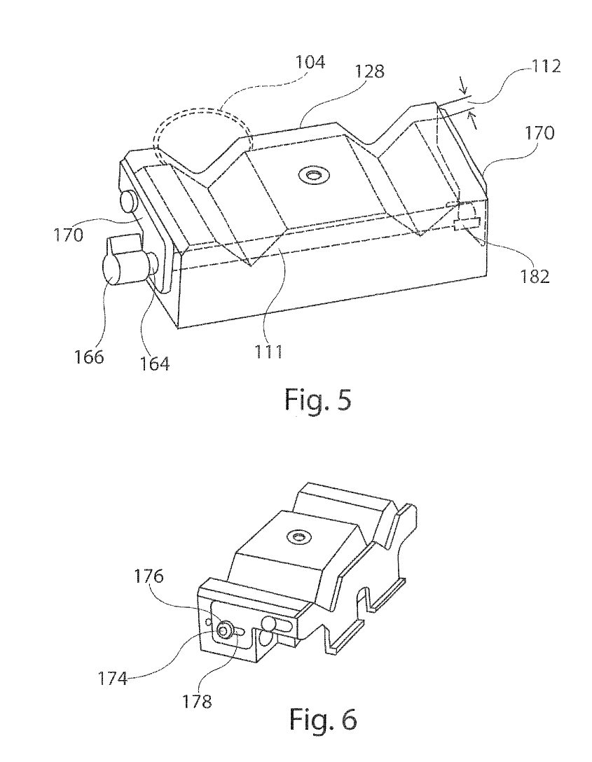

Referring now to FIGS. 5-6, the syringe pump also includes a quick connect/disconnect syringe barrel and syringe barrel flange capture assembly. The syringe barrel flange capture assembly will first be described. The barrel flange capture assembly includes a barrel flange capture bracket 128 which is provided with tabs 170 which in turn are slidably mounted in a locking shaft 110.

Locking shaft 110 is provided with a locking nut 164 and a threaded thumb screw or knob 109. The shaft 110 passes through tabs 170 on the syringe barrel flange capture bracket 128 and then through syringe block 106. The distal end 174 of the shaft 110 opposite the thumb-screw or knob 109 includes a flange 176 and retaining clip 178 or other similar device so that as the thumb-screw or knob 109 is turned, the side tabs 170 of the flange capture bracket 128 are pulled tightly against the syringe block 106, locking the assembly in place. Alternatively, knob 109 may be replaced by a rocker cam. The shaft 110 may be provided with an anti-rotation 182 devices to eliminate rotation of the shaft 110 as the thumb-screw or knob 109 is turned, thus further facilitating the quick connect/disconnect tool-free locking procedure. While the syringe barrel flange capture bracket 128 is unlocked, it may be slidably adjusted with respect to the syringe block 106, providing an adjustable gap 112. The adjustable gap 112 may be set to fit a variety of syringe barrel flanges 104 associated with different types and sizes of syringes available for use with the syringe pump.

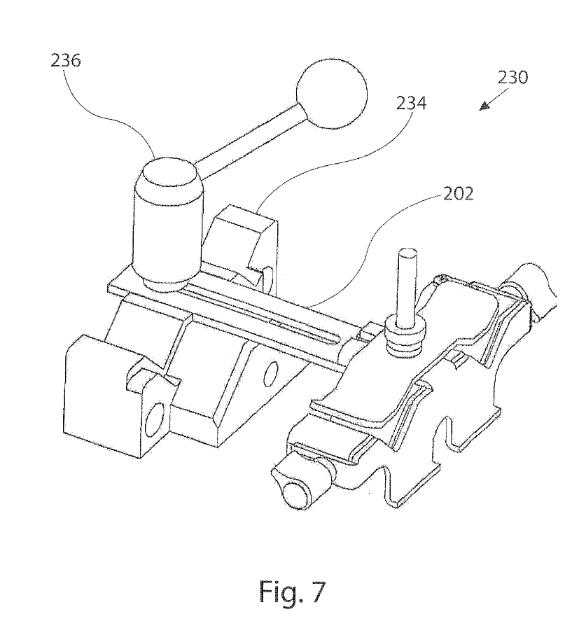

Referring now to FIG. 7, and yet another mechanical improvement, there is provided a bar and clamp for mounting to a syringe pump for facilitating accommodation of syringes of different sizes, and/or for permitting simultaneous infusion and withdrawal of liquids from a pair or more of syringes whose plungers are mounted fixedly on opposing faces of a moveable carriage. More particularly, as shown in FIG. 7, a syringe holder and clamp assembly 230 is mounted on one end of an adjustable extension bar 202 that is attached to the main block 234 with a mechanical fastener 236 such as a twist nut or cam-lock. The main block 234 is rigidly attached to the syringe pump, i.e. and replaces block 50 as shown in FIG. 2. The syringe holder and clamp assembly 230 may be mounted with the extension bar 202 extending to either side and is slidably adjustable with respect to the main block 234 to accommodate a variety of syringes and syringe sizes that may be used in conjunction with a syringe pump whose overall length is fixed by design. In this manner, essentially any size syringe and/or combinations of syringes of different sizes may be used in a pumping configuration. Also, a pair or more of syringes may be fixedly mounted on a single syringe pump permitting simultaneous infusion and withdrawal of liquids.

Summarizing to this point, the present invention provides several mechanical improvements in syringe pumps. These include, in no particular order of importance: In one aspect of the invention there is provided a syringe pump in which a pusher block is slidably mounted on bearings on one or more guide rails, wherein the pusher block is advanced or retracted by a lead screw having an axis which is offset from the axis of the one or more guide rails which are supported at their respective ends by support blocks. Improved resistance to torque is achieved by extending the forward and/or trailing sides of the bearings, wherein the guide rails support blocks include recesses for accommodating the extended ends of the carriage slide bearings.

The invention also provides a syringe pump having a tool-free quick connect/disconnect facilitates loading and unloading of syringes from the pump. In one embodiment, the quick connect/disconnect comprises a slidably adjustable syringe plunger flange capture bracket, and preferably includes a locking cam and nut, and/or a threadable knob for drawing side tabs of the flange capture bracket against the pusher block. In another embodiment, the quick connect/disconnect comprises a slidably adjustable syringe barrel flange capture bracket, and preferably includes a locking cam and nut, and/or a threadable knob for drawing side tabs of the flange capture bracket against the syringe block.

Yet another aspect of the invention provides a syringe pump in which a pusher block is slidably mounted on one or a plurality of guide rails, and further including a tool-free quick connect/disconnect syringe barrel clamp.

The present invention also provides a syringe pump in which a pusher block is a carriage which is slidably mounted on one or a plurality of guide rails, and including a syringe holder and clamp mounted on an extension bar, wherein the syringe holder and clamp is slidably adjustable with respect to the pusher block to accommodate a variety of syringes and syringe sizes.

It is thus seen that the present invention provides several mechanical enhancements by the improved performance and reliability, convenience and flexibility over current syringe pumps.

Yet other improvements over current syringe pumps will be described below.

### Programmable Control Improvements

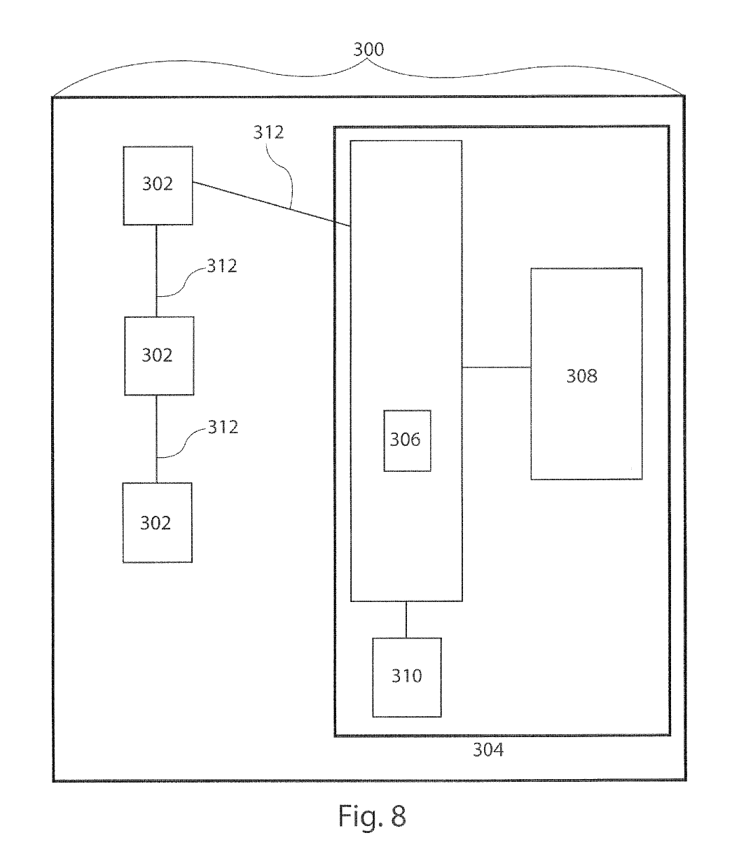

Referring to FIG. 8, in yet another aspect of the invention, there is provided a master-satellite syringe pumping system 300 which comprises one or more satellite syringe pumps 302 controlled and powered by a separate stand-alone master pump 304. The master pump 304 includes a control system 306 having microprocessor for storing run protocols, and including a graphical user interface (GUI) 308 for permitting user input and for displaying user information, and a power supply 310 which provides, as needed, electrical power for the operation of one or more satellite syringe pumps 302. Cables 312 containing a plurality of conductors are used to distribute electrical power and control signals, as needed, and as a pathway for communication between the master pump 304 and each of the satellite units 302. With this arrangement, a user may adjust and set any or all master/satellite pump operation parameters from the master pump 304 control 306, without the need for a PC or other stand-alone control system.

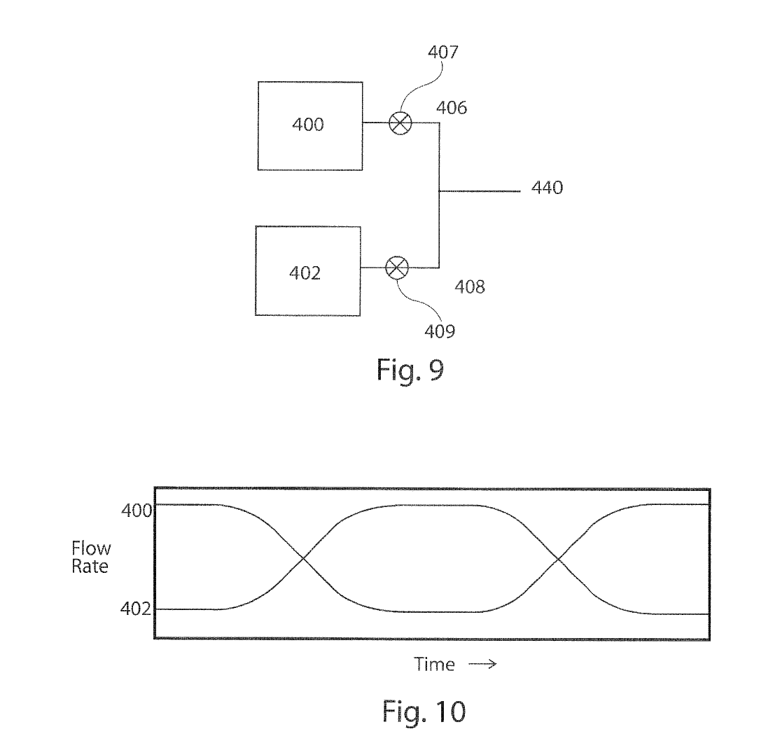

Referring now to FIGS. 9 and 10, yet another improvement, provides better flow rate control when delivering fluid from multiple syringes into a single flow channel. The present invention provides a way of operating a pair (or more) of syringe pumps in a coordinated and sequential order, and combining their output into a single flow channel. More particularly syringes 400, 402 are connected via tubing 406, 408 and valving 407, 409 into a single main distribution tube 410. Flow from each syringe is adjusted to contribute to a total flow rate desired in the main distribution tube 410. The overall process is as follows: fluid from the first syringe 400 is set at a desired flow rate. At a predetermined time before the volume of fluid contained in the first syringe 400 is depleted, the flow rate from the first syringe 400 is reduced, while the flow rate from a second syringe 402 is increased to match the reduction in flow rate from the first syringe 400. Flow volumes from the first and second syringes 400, 402 are adjusted until the flow rate from the first syringe 400 is at zero, while the flow rate from the second syringes 402 is adjusted to the desired flow rate. The first syringe 400 may then be replaced with a fresh filled syringe, or the first syringe may be refilled and the cycle repeated. In this manner, perturbations of flow are minimized at the cross-over between syringes. This method may be used to either infuse or withdraw fluids through the main distribution tube 410, resulting in a smooth flow rate. This improvement is particularly useful in connection with liquid chromatography where multiple pumps may be employed, and continuously smooth flow is desired. This method of crossover correction may be used with 2, 3, 4 etc. pumps with combined flow.

Yet other embodiments are possible. For example, pump flows may be controlled to vary, for example, by pulsing so as to mimic physiological characteristics such as blood flow, or to mimic a physiological waveform such as, for example, an EKG through a dispense profile. This latter feature is particularly useful where syringe pumps are being used to administer a physiological fluid, for example, to a laboratory animal. This also may use 1, 2, 3, 4 or more syringe pumps.

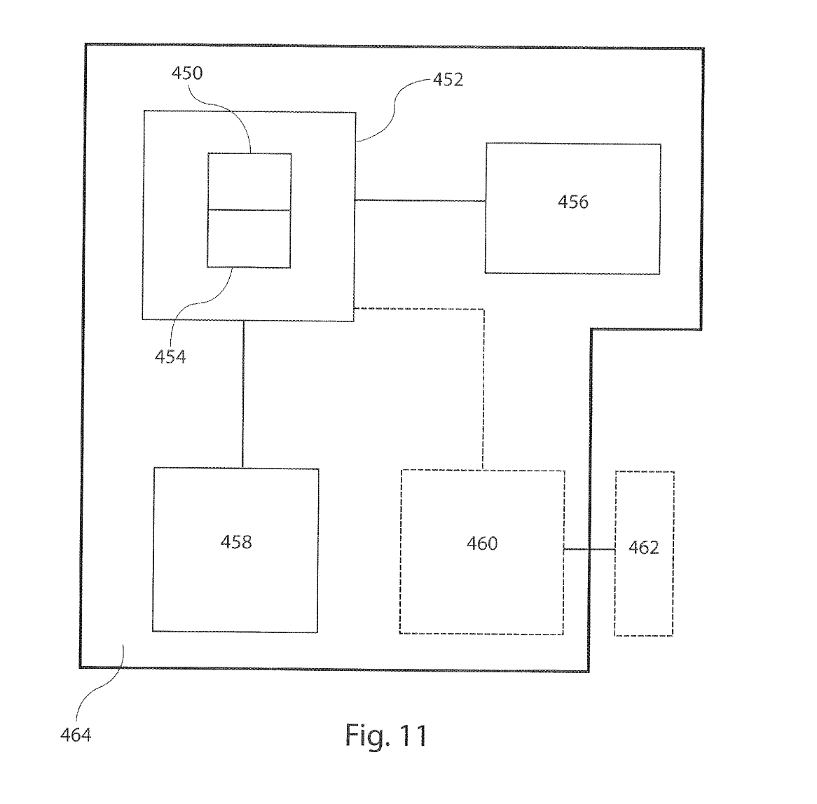

In yet another aspect of the invention, illustrated in FIG. 11, pumping protocols are stored in an electronic library 450, and the syringe pump controlled by an electronic control system 452 to run at a pre-programmed speed, or other variable. Other variables stored in the library include, for example, any and all parameters associated with the operation of the pump and experiment: syringe capacity, pressure, force of syringe, syringe diameter, syringe strength, syringe length, temperature, date, time, operator, approval levels to operate, languages, etc. In a preferred embodiment, electronic control system 452 contains a processor operably coupled to a memory 454, a motor driver circuit 456 and a graphical display system 458 whose user interface imagery is controlled by the processor via software and firmware stored in memory. Actuation force may be controlled by the pump operator using one of two methods. In one method, the pump operator, through the graphical display system or a PC controlled software terminal, may select a force rating equivalent to a percentage of the maximum possible actuation force of the syringe pump. In another method, the pump operator may select a syringe, which calls for a default force rating which has been pre-stored in a software library and which corresponds to a specific syringe. The library may contain an array of characteristics for a given syringe, including volumetric capacity, internal diameter and maximum safe applied force. Maximum safe applied force is an operational control limit of applied force above which level may result in damage to the syringe. In either method, the electronic control system is tasked with regulation of electrical current to the stepper motor or other motor. Consequently, torque generated by the stepper motor may be controlled, and thus force applied to the syringe plunger as the motor torque is translated into a linearly-directed force through the leadscrew and half-nut or full-nut or rack and pinion. The half-nut or full-nut or rack and pinion is connected in a rigid manner to the linear slide mechanism, so all forces applied upon the half-nut or full-nut are transferred directly to the linear slide mechanism and finally to the syringe plunger. In a preferred embodiment the system also may include a bar code reader a RFID reader, IR reader, or other reader 460 for identifying the particular syringe 462 as the syringe is placed in the pump 464. The output from the reader is then transmitted to the processor identified by the electronic library 450, and the control set.

Volume of the syringe can be recorded in this table and can be read by an ultrasonic signal directed down the syringe and measured by reflections back to a sensor which will read actual volume in the syringe. The volume measurements may be stored in the library, and updated as volume varies in the pump/experiment.

Also, if desired, the syringe pump may be provided with sensors and programs to adjust for temperature, pressure feedback sensors, and the like. Additionally, the syringe pump may be controlled by a processor which includes a memory having a library of selectable user interfaces, etc which permits an operator to input variables (material, syringe size, etc.) and then choose from a library of pre-assigned test protocols or the like.

Summarizing, the present invention also provides several programming and control improvements for operating syringe pumps. These include, in no particular order of importance:

In one aspect of the invention, there is provided a syringe pump control system comprising a master pump and one or more satellite syringe pumps, in which the satellite syringe pumps are controlled and powered by the master pump, and in which the control system electrical power and controls for operation of the one or more satellite pumps are distributed from the master pump. Preferably, the master pump includes a control system having a memory for storing and controlling run protocols.

The invention also provides a method for maintaining smooth and consistent flow rates in a single flow channel, when the total flow rate is comprised of the sum of flows eminating from at least two syringes operated in a coordinated and sequential order. With such method it is possible to remove and replace or refill one of syringe while the other or others are dispensing or withdrawing fluid at a predetermined flow rate.

In another aspect of the invention, one or more syringe pumps are pulsed to mimic physiologic characteristics, e.g., an EKG, or blood flow.

The present invention also includes a microprocessor controlled syringe pump system in which syringe actuation forces are controlled through look-up table in a computer library, which in a preferred embodiment includes pre-assigned test protocols.

In yet another aspect of the invention the syringes carry bar codes or RFID tags, or IR tags, and the pump includes a bar code reader, RFID reader or IR reader for identifying a syringe and transmitting information regarding the syringe to the microprocessor.

In yet another aspect an encoder will mark rotational position of the lead screw, while another encoder will read the travel of the pusher block at a set rate. The theoretical time and distance will be calculated and compared to actual. These values will be stored in a library. During a run the theoretically stored values will be compared to actual and fed into a motor control circuit to increase or decrease block movement and screw turning to more closely achieve theoretical time and distance. This will result in a smooth flow with minimal pulsings and high accuracy and precision. The encoder will insure accurate mapping irrespective of where the block starts.

The above invention has been described in connection with syringe pumps. However, several of the above described features may have utility outside of the syringe pump field. For example, the flow rate control shown in FIGS. 9 and 10 may be used in other flow control applications.

Yet other features and advantages of the invention will be apparent to one skilled in the art, taking into account the above description.

1. A syringe pump in which a pusher block is slidably mounted on bearings on one or more guide rails, wherein the pusher block is advanced or retracted by a lead screw having an axis which is offset from the axis of the one or more guide rails which are supported at their respective ends by support blocks, wherein forward and/or trailing sides of the pusher block bearings are extended, and wherein the guide rail support blocks include recesses for accommodating the extended ends of the carriage slide bearings.

2. A syringe pump in which a pusher block is slidably mounted on one or a plurality of guide rails, the improvement which comprises a set of tool-free quick connect/disconnect for facilitating loading and unloading of syringes from the pump.

3. The syringe pump as claimed in claim 2, wherein the quick connect/disconnect comprises a slidably adjustable and lockable syringe flange capture bracket.

4. The syringe pump as claimed in 2, wherein the quick connect/disconnect includes a thumb-screw, knob or locking cam for drawing side tabs of the flange capture bracket against either a pusher block or a syringe holder block.

5. The syringe pump as claimed in claim 4, further including an anti-rotation device to the quick connect/disconnect with prevents rotation of the shaft as the thumb-screw or knob is turned.

6. A syringe pump in which a pusher block is slidably mounted on one or a plurality of guide rails, and further including a tool-free quick connect/disconnect syringe barrel clamp.

7. A syringe pump in which a pusher block is slidably mounted on one or a plurality of guide rails, and including a syringe holder and clamp mounted on an extension bar, wherein the syringe holder and clamp is slidably adjustable with respect to the pusher block to accommodate a variety of syringes and syringe sizes.

8. A system of syringe pumps comprising a master pump and one or more satellite syringe pumps, wherein the satellite syringe pumps are controlled and powered by the master pump, and wherein electrical power and controls for operation of the one or more satellite pumps are distributed from the master pump.

9. The syringe pump as claimed in claim 8, wherein the master pump includes a control system having a memory for storing run protocols.

10. A method for maintaining smooth and consistent flow rates to and from a syringe pump, which comprises output from at least two syringes into a single flow channel, and operating the at least two syringes in a coordinated and sequential order in order to maintain a consistent flow rate or pressure.

11. The method of claim 10, and including removing and replacing or refilling one of the syringe pumps while the other or others are dispensing or withdrawing fluid at a predetermined flow rate.

12. The method of claim 11, wherein the syringes dispense fluid at a constant volume flow rate, or constant pressure or constant force.

13. The method of claim 11, wherein the syringes withdraw fluid at constant volume flow rate, or constant pressure, or constant force.

14. A method of operating one or more syringe pumps which comprises pulsing the syringe pumps to mimic physiologic characteristics.

15. The method of claim 14, wherein the physiological characteristic comprises an EKG.

16. The method of claim 15, wherein the physiological characteristic mimics blood flow.

17. A syringe pump system in which syringe actuation forces are controlled through look-up table in a computer library.

18. The syringe pump system of claim 17, further including an interface for permitting a user to manually select syringe actuation force.

19. The syringe pump system of claim 17, further including a bar code, RFID, IR or other reader for identifying a syringe and transmitting information regarding the syringe to the system.

20. The syringe pump system of claim 19, wherein the library includes pre-assigned test protocols.

21. A method for controlling a syringe pump, which comprises comparing actual syringe movement to a theoretical desired value, and adjusting said syringe movement to approximate said theoretical value.

22. The method of claim 21, wherein said syringe is moved by a screw mechanism, and including the step of adjusting rotation of said screw.

23. The method of claim 22, wherein said screw profile is determined by one or more encoders.
